(19) United States
(12) Patent Application Publication
(10) Pub. No.: US 2019/0358453 A1
(43) Pub. Date: Nov. 28, 2019

(54) GALVANIC SKIN RESPONSE DETECTION WITH CRANIAL MICRO DIRECT CURRENT STIMULATION

(71) Applicant: Desire DUBOUNET, Budapest (HU)

(72) Inventor: Desire DUBOUNET, Budapest (HU)

(21) Appl. No.: 16/348,645

(22) PCT Filed: Jul. 7, 2017
(86) PCT No.: PCT/HU2017/000043

§ 371 Date: May 9, 2019

(30) Foreign Application Priority Data
Nov. 9, 2016 (HU) P1600614

(51) Int. Cl. A61N 1/36; A61B 5/053; A61B 5/00; A61N 1/22; A61N 1/04

(52) U.S. Cl. CPC A61N 1/36031; A61B 5/0533; A61B 5/6803; A61B 5/6898; A61B 5/6822; A61N 1/0456; A61N 1/0476; A61N 1/36034; A61N 1/22

(57) ABSTRACT

Disclosed is a process whereby galvanic skin response is measured after the stimulation of the cranium by micro direct current. The body is stimulated through the cranium by micro direct current by creating a cybernetic loop of a loop of current-carrying material, after which the body's responses are measured. The process uses a galvanic skin response and voltage measuring and frequency counting unit; this galvanic skin response data is then used in the same process cycle to generate secondary stimulation. The cybernetic loop is attached to the upper part of the cranium with three connection points to the upper forehead. All parts are connected with the electrode coil, consisting of five clockwise and five counter-clockwise loops, the silver necklace around the neck used as grounding, and the appropriate jack outputs. The method uses the input signals processed by the control software and the emitted micro direct current.

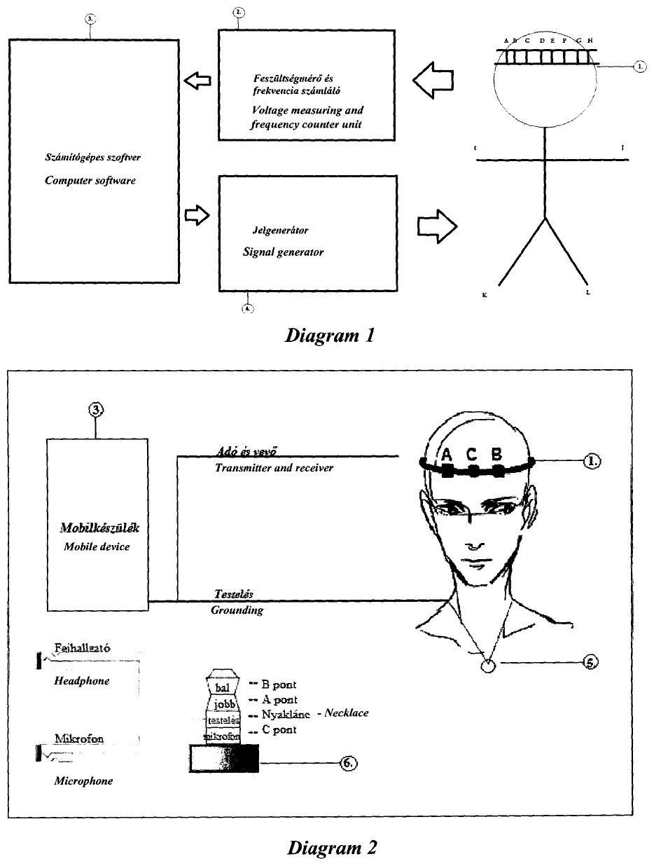

The subject of the patent is a process whereby the body is stimulated through the cranium by micro direct current with a current-carrying loop installed around the cranium. The induced response of the body is then measured and processed by, among other things, measuring the galvanic skin response.

The beneficial effect of electrotherapy and electric current on the body has been known for nearly 2000 years and is a science that has been the subject of experiments, as shown by a vast available literature from ancient times. Low-intensity electric stimulation of the body is considered by the general public as a kind of a foundation of research throughout the ages. Even though later on the effects of electric current was tested by using galvanic cell, it was still a subject on unceasing attention in the middle ages. It was pursued by Giovanni Aldini and Alessandro Volta, and by many others during the 18^{th} century.

In the '60s and '70s, it was an approved method of treatment among physicians and researchers to place electrodes on the eyes and use microampere current, which was considered to be the most efficient—although not the only—way to send electric current to the brain through the cranium. Later on, it was discovered that instead of this bizarre method, a much more efficient treatment is to use electrodes on the forehead.

The foundation of the research and studies on the stimulation of the cranium by electric current is to test the effects it has on the body, especially focusing on the treatment of insomnia, and it has been given a variety of descriptions throughout the years, such as “transcranial electric therapy” or “electrostimulation”. One way to conduct a treatment based on transcranial electric stimulation is that the heart rate, which is increased by the electricity, stimulates serotonin production in the nerve cells while also stimulating dopamine and endorphin production, as well as influencing other hormones that are beneficial to the nervous system and improve the synapsis between nerves.

Current research seems to indicate that increasing the levels of serotonin, norepinephrine and dopamine in the brain will proportionally reduce the level of cortisol. After a treatment of low-current stimulation of the cranium, the subjects in the research have described a calm but alert state as their experience. EEG testing during the treatment shows an increase in alpha brain waves and a decrease in delta brains waves. Thus, we see that the interactions between the body and electricity engages a broad field of research. Let us introduce a number of patents relevant to the field: the patent titled “MODIFICATION OF THE GROWTH, REPAIR AND MAINTENANCE BEHAVIOR OF LIVING TISSUES AND CELLS BY A SPECIFIC AND SELECTIVE CHANGE IN ELECTRICAL ENVIRONMENT”, registered under patent number U.S. Pat. No. 4,315,503, is a surgically non-invasive method of inducing voltage in tissue and specifically in cells to convey impulses to the human body. Contact with the body is established in the area between the ankle and the knee. Another procedure that is closer to the brain, but whose long-term application is very dangerous, is the patent titled “TRANSCRANIAL ELECTROSTIMULATION DEVICE AND METHOD”, patent number U.S. Pat. No. 9,186,505, which is a transcranial—meaning it is performed through the skull—electrostimulation process used for the management of depression and pain. The description of the process mentions, among others, a white noise clock generator and voltage adjustment. However, long term use can lead to hyperpolarization, constituting a safety risk. The patent registered under No. CN105107085 and titled “MOBILE EQUIPMENT, BASED CRANIAL ELECTROTHERAPY STIMULATION INSTRUMENT” is a mobile equipment based instrument and process which comprises a microcontroller unit and a stimulation circuit, managed by a human controller through a data port. The invention indicates an initial concept that does not rule out therapeutic use, although the inventor does not mention any detailed information on this aspect. A similar invention to the one above is the “CLOSED LOOP TRANSCRANIAL DIRECT CURRENT STIMULATION DEVICE”, registered under No.

CN105013080, which creates a closed loop of direct current around the head, involving a signal acquisition and emitting apparatus. The device also contains a microfilament electrode and a pre-differential amplifier. It is adjusted to the head by a cap where the electric output is transmitted. As described, the device is very complicated and signal generation is also rather complex, increasing the possibility or errors with its elaborate composition. The description does not mention the benefits or scope of use for the invention, it only informs us in detail on the architecture of the device. Although these inventions involve electric stimulation, the issues they attempt to solve and the implementation they imply follows a different train of thought than the solution in our submitted process. The patent “TRANSCRANIAL PULSED CURRENT STIMULATION”, registered under US2016136424, similarly to the previous inventions, is a computer-implemented method. It uses electrodes to apply pulses to the brain and a pre-written electrotherapy stimulation program to apply voltage. This process uses voltage as well, in the shape of rectangular voltage signals. Due to its specific voltage signal basis, I do not consider it relevant for my invention. However, citing it provides further confirmation for the rationale of this field of research, meaning that countless researchers are working on cranial electrostimulation and the limits of the field of research is practically endless. Still, these researchers all have a different approach to the problem with non-overlapping solutions, thanks to the yet unexploited issues and possible answers of the field.

Our process is called “GSRtDCs” (galvanic skin response—transcranial direct current stimulation), and the name basically describes the steps of the process (measuring galvanic skin response by stimulating the skull with micro direct current). One of our objectives is for the controller and evaluation unit of the above process to be available as a software for smart phones and computers. Low-voltage current, as the case may be, can be connected to the body through the headphone jack of a smartphone at the upper part of the forehead. Then, the response we want to measure is received through the microphone out jack of the smartphone, where the software independently evaluates, records, and uses it for determining the next output signals. In this case, this is a cycle that returns to itself, meaning that the response induced by the output signal is used within the same process for creating the output signal of the next process, determining its strength and duration.

Our invention is therefore based on an application that can run on a phone or a computer. Signals are sent and received through the outgoing port of the headphone. The output attached to the jack creates a connection between the body and the loop around the upper part of the cranium and operates by measuring the evoked response reaction.

In order to measure the evoked potential difference induced by electric current, we naturally use only safe, low voltage direct current, making this technology completely harmless to the human body where no damages may incur.

Basically, we are working with a natural biological response reaction that allows us to analyse the processed details.

We create an impulse primarily through a mobile phone, although the system also works on a mobile connected to a computer and on a standalone computer as well. In any two devices, a pre-installed software (application) manages and controls the process.

The process involves direct current connecting to the body through the upper part of the forehead. Safety is best ensured by using a silver necklace around the neck as the body point, or a graphite-covered band placed around the ankle. Due to creating a body point, the brain functions of the treated individual are not sedated; on the contrary, brain functions increase.

Through the beneficial implementation of our solution, the potential of the outgoing branch is controlled to fall between 0-4 Volt, a value below the human sensory threshold. This is coupled with 0-7 mA to guarantee safety and avoid any inconvenient sensation during treatment.

The method is basically a cyclical process that measures the body's response to an output signal with various devices (detailed below), where data is processed by a central unit, which not only stores the incoming signal but uses it to produce a new signal. Measurement data are recorded during the entire process. The method produces data that can later on be used in other processes and treatments. All our experiments described thus far have been conducted with a current-carrying loop where the body's electrically evoked reactions and stimuli are processed.

Our goal is to stimulate the body by using a current-carrying loop and micro direct current to the cranium, measuring skin resistance (using measurements of galvanic skin response) to calculate the parameters for the next output of micro-ampere stimulation. It was our primary objective to make this program work on a telephone or a simple commuter. With this method, due to the unique measurements, we generate data that are suitable to support certain treatments and processes later on. This is to say that our method, in and of itself, is not a therapeutic method; it is primarily a unique method to generate and collect data for the benefit of other methods and further research. Although a great number of research materials deal with the beneficial physical and mental changes experienced during and after the process, but it was our primary goal to find a unique solution to generate data, for which we found the present process to be the most satisfying.

When implementing the method, we use the output of a low-voltage current through the headphone jack of an arbitrary mobile phone by attaching a loop to the upper part of the forehead to carry the current. We collect and store the data of the body's response to the impulse outputs through the microphone jack of the device. New impulses of micro current are generated by analysing the values collected through measuring the galvanic skin response, so its values are not fixed during the process.

In order to measure galvanic skin response, we need to apply a voltmeter. This makes it possible to measure the body's reactions through skin response as it reacts to the electric output signal. Since today's mobile phones are able to generate and receive electric signals, they are perfect tools to implement the discusses method, while the installed software can make changes to the hardware of the device.

The current-carrying loop mentioned above is a cybernetic loop, basically an electrode that we feed with electric stimulation by running the software, where the outlet of the loop is connected to the headphone jack and signals are emitted through the jack (headphone-speaker unit). Returning signals are received through the appropriate receiving unit (microphone unit) for further analysis. By this, we create an electrode called the cybernetic loop that independently transmits the signals/waves evoked for the treatment and receives them for analysis and to generate the signal for evoking the next response. It is important that the electrode coil is applied to the upper part of the cranium in five clockwise loops and five counter-clockwise loops to evoke the expected transcranial effect and to generate a scalar field. The electrode touching the upper part of the forehead has three sensors made of graphite-covered rubber to transmit the signals. Furthermore, both electrode coils are grounded to a silver necklace worn around the neck.

Hereunder, we wish to describe the process for which registration is sough and its processes by using schematic diagrams to make comprehension easier.
Diagram 1: Theoretical operational process
Diagram 2: Schematic diagram of jack allocation

Diagram 1 is a sketch of the operational process of the method. In the first step, a loop made of current-carrying material is attached to the upper part of the head on the forehead. This loop is called the cybernetic loop, which is a coil created by five clockwise loops and five counter-clockwise loops to evoke the expected field strength. A cycle process is carried out with this method, using the signals emitted and received through the microphone and headphone jacks of a device suitable for running the control software 3. Thereby, the signal determined by the software and emitted by the signal generator 4 travels through the headphone jack to reach the 1 loop made of current-carrying material and leaves through the forehead at the attachment points. Detectable changes evoked by the stimulation of the body are processed and recorded by the 2 voltage measuring and frequency counting unit, after which the cycle restarts but new signals are now different from the one emitted previously, determined by the incoming data. Diagram 2 depicts the jack allocation most suitable for the method, indicating that the 1 loop made of current-carrying material with its three connection points and the grounding on the neck in the form of a silver necklace is connected to the 6 jack allocation, and thereby to the 3 (mobile) device running the control software.

### LIST OF REFERENCE SIGNS

1—Loop made of current-carrying material
2—Voltage measuring and frequency counting unit
3—Software control
4—Signal generator
5—Grounding
6—Jack allocation

1-10. (canceled)

11. A method for measuring galvanic skin response through direct current cranial stimulation, comprising: stimulating the body through the skull by micro direct current with the help of a loop (1) made of current-carrying material, after which the body's response is measured, recorded and used, with the help of a galvanic skin response and voltage measuring and frequency counting unit (2) and the data processed by a control software (3), where said data is used for further signal output in the cycle to determine the adequate signal.

12. The method specified in claim 11, wherein the cycle-process being directed by an evaluation and control software (3) that runs on a mobile phone or computer, the output micro current is connected to the body through the headphone jack with the help of a loop made of current-carrying material and placed on the upper part of the forehead, and signal output and data for measuring the response is transmitted to the control software (3) by a compatible jack.

13. Equipment required for the process specified in claim 11, comprising a loop made of current-carrying material (1) being a cybernetic loop, the unique feature of which is that it is applied to the upper part of the cranium in five clockwise loops and five counter-clockwise loops to evoke the expected parameters, and where the electrode touching the upper part of the forehead has three sensors made of graphite-impregnated rubber and the neck section is connected to a silver necklace as grounding, which may be substituted by graphite-covered bands on the ankle.

14. Equipment required for the process specified in claim 11, wherein the body point placed on the neck is a silver necklace attached to the microphone and headphone jack allocations.

15. The method specified in claim 11, wherein the transcranial stimulation uses solely the signals created by a signal generator (4) through the control software, and wherein the evoked responses are measured and processed and the data thus elicited can be used in developing other research and processes.

16. The method and its equipment specified in claim 11, wherein the output is solely voltage between 0 and 4 Volts and the current is between 0 and 7 mA to guarantee that no harm is done to the subject during the process.

17. The method specified in claim 11, wherein the method is performed using a mobile device or a computer to process and emit signals, which is connected through a standard jack plug output to the loop made of current-carrying material attached to the head (1) and the necklace serving as grounding on the body (5).

18. Equipment required for the process specified in claim 13, wherein the body point placed on the neck is a silver necklace attached to the microphone and headphone jack allocations.

19. The method and its equipment specified in claim 12, wherein the output is solely voltage between 0 and 4 Volts and the current is between 0 and 7 mA to guarantee that no harm is done to the subject during the process.

20. The method and its equipment specified in claim 13, wherein the output is solely voltage between 0 and 4 Volts and the current is between 0 and 7 mA to guarantee that no harm is done to the subject during the process.

21. The method and its equipment specified in claim 14, wherein the output is solely voltage between 0 and 4 Volts and the current is between 0 and 7 mA to guarantee that no harm is done to the subject during the process.

22. The method and its equipment specified in claim 15, wherein the output is solely voltage between 0 and 4 Volts and the current is between 0 and 7 mA to guarantee that no harm is done to the subject during the process.

23. The method and its equipment specified in claim 18, wherein the output is solely voltage between 0 and 4 Volts and the current is between 0 and 7 mA to guarantee that no harm is done to the subject during the process.
